SEQUENCE LISTING (7 sequences)

SEQ ID NO: 1 - protein, 307 aa, Homo sapiens
MAALLLGAVLLVAQPQLVPSRTAELGQQELLRKAGTLQDDVRDGVAPNGSAQQLPQTIIIGVRKGGTRALLEMLSLHPDVAAAENEVHFFDWEEHYSHGLGWYLSQMPFSWPHQLTVEKTPAYFTSPKVPERVYSMNPSIRLLLILRDPSERVLSDYTQVFYNHMQKHKPYPSIEEFLVRDGRLNVDYKALNRSLYHVHMQNWLRFFPLRHIHIVDGDRLIRDPFPEIQKVERFLKLSPQINASNFYFNKTKGFYCLRDSGRDRCLHESKGRAHPQVDPKLLNKLHEYFHEPNKKFFELVGRTFDWH

SEQ ID NO: 2 - protein, 367 aa, Homo sapiens
MAYRVLGRAGPPQPRRARRLLFAFTLSLSCTYLCYSFLCCCDDLGRSRLLGAPRCLRGPSAGGQKLLQKSRPCDPSGPTPSEPSAPSAPAAAVPAPRLSGSNHSGSPKLGTKRLPQALIVGVKKGGTRAVLEFIRVHPDVRALGTEPHFFDRNYGRGLDWYRSLMPRTLESQITLEKTPSYFVTQEAPRRIFNMSRDTKLIVVVRNPVTRAISDYTQTLSKKPDIPTFEGLSFRNRTLGLVDVSWNAIRIGMYVLHLESWLQYFPLAQIHFVSGERLITDPAGEMGRVQDFLGIKRFITDKHFYFNKTKGFPCLKKTESSLLPRCLGKSKGRTHVQIDPEVIDQLREFYRPYNIKFYETVGQDFRWE

SEQ ID NO: 3 - protein, 393 aa, Homo sapiens
MAPSGPTGAQPSPAEPLSRSIFRKFLLMLCSLLTSLYVFYCLAERCPPGSGPVAGVPGRGVPAGPRELAMWPAGAPRKRLLQLRQRRRRGRSGPGDSSDQEEQSPGLAAAPGGSGAGSSVAEAQPGTLALLLDEGSKQLPQAIIIGVKKGGTRALLEFLRVHPDVRAVGAEPHFFDRSYHKGLAWYRDLMPRTLEGQITMEKTPSYFVTREAPARISAMSKDTKLIVVVRDPVTRAISDYTQTLSKRPDIPSFESLTFRNRSAGLIDTSWSAIQIGLYAKHLEPWLRHFPLGQMLFVSGERLVSDPAGELRRVQDFLGLKRIITDKHFYFNQTKGFPCLKKAEGSGKPHCLGKTKGRAHPTIAREVLRQLRDFYRPFNRKFYQMTGRDFGWDG

SEQ ID NO: 4 - protein, 390 aa, Homo sapiens
MGQRLSGGRSCLDVPGRLLPQPPPPPPPVRRKLALLFAMLCVWLYMFLYSCAGSCAAAPGLLLLGSGSRAAHDPPALATAPDGTPPRLPFRAPPATPLASGKEMAEGAASPEEQSPEVPDSPSPISSFFSGSGSKQLPQAIIIGVKKGGTRALLEFLRVHPDVRAVGAEPHFFDRSYDKGLAWYRDLMPRTLDGQITMEKTPSYFVTREAPARISAMSKDTKLIVVVRDPVTRAISDYTQTLSKRPDIPTFESLTFKNRTAGLIDTSWSAIQIGIYAKHLEHWLRHFPIRQMLFVSGERLISDPAGELGRVQDFLGLKRIITDKHFYFNKTKGFPCLKKAEGSSRPHCLGKTKGRTHPEIDREVVRRLREFYRPFNLKFYQMTGHDFGWD

SEQ ID NO: 5 - protein, 456 aa, Homo sapiens
MARWPAPPPPPPPPPPLAAPPPPGASAKGPPARKLLFMCTLSLSVTYLCYSLLGGSGSLQFPLALQESPGAAAEPPPSPPPPSLLPTPVRLGAPSQPPAPPPLDNASHGEPPEPPEQPAAPGTDGWGLPSGGGGARDAWLRTPLAPSEMITAQSALPEREAQESSTTDEDLAGRRAANGSSERGGAVSTPDYGEKKLPQALIIGVKKGGTRALLEAIRVHPDVRAVGVEPHFFDRNYEKGLEWYRNVMPKTLDGQITMEKTPSYFVTNEAPKRIHSMAKDIKLIVVVRNPVTRAISDYTQTLSKKPEIPTFEVLAFKNRTLGLIDASWSAIRIGIYALHLENWLQYFPLSQILFVSGERLIVDPAGEMAKVQDFLGLKRVVTKKHFYFNKTKGFPCLKKPEDSSAPRCLGKSKGRTHPRIDPDVIHRLRKFYKPFNLMFYQMTGQDFQWEQEEGDK

SEQ ID NO: 6 - protein, 346 aa, Homo sapiens
MLFKQQAWLRQKLLVLGSLAVGSLLYLVARVGSLDRLQPICPIEGRLGGARTQAEFPLRALQFKRGLLHEFRKGNASKEQVRLHDLVQQLPKAIIIGVRKGGTRALLEMLNLHPAVVKASQEIHFFDNDENYGKGIEWYRKKMPFSYPQQITIEKSPAYFITEEVPERIYKMNSSIKLLIIVREPTTRAISDYTQVLEGKERKNKTYYKFEKLAIDPNTCEVNTKYKAVRTSIYTKHLERWLKYFPIEQFHVVDGDRLITEPLPELQLVEKFLNLPPRISQYNLYFNATRGFYCLRFNIIFNKCLAGSKGRIHPEVDPSVITKLRKFFHPFNQKFYQITGRTLNWP

SEQ ID NO: 7 - protein, 341 aa, Homo sapiens
MAGSGGLGGGAGGGQGAGAGQGAALRASRAPMLLVALVLGAYCLCALPGRCPPAARAPAPAPAPSEPSSVHRPGAPGLPLASGPGRRRFPQALIVGVKKGGTRALLEFLRLHPDVRALGSEPHFFDRCYERGLAWYRSLMPRTLDGQITMEKTPSYFVTREAPRRIHAMSPDTKLIVVVRNPVTRAISDYAQTLSKTPGLPSFRALAFRHGLGPVDTAWSAVRIGLYAQHLDHWLRYFPLSHFLFVSGERLVSDPAGEVGRVQDFLGLKRVVTDKHFYFNATKGFPCLKKAQGGSRPRCLGKSKGRPHPRVPQALVRRLQEFYRPFNRRFYQMTGQDFGWG

(19) United States
(12) Patent Application Publication
(10) Pub. No.: US 2019/0086428 A1
(43) Pub. Date: Mar. 21, 2019

(54) ALZHEIMER'S DISEASE EARLY DIAGNOSIS AND/OR PROGNOSIS IN CIRCULATING IMMUNE CELLS BASED ON HEPARAN SULFATES AND/OR OF HEPARAN SULFATE SULFOTRANSFERASES

(71) Applicants: Universite Paris Est Creteil Val De Marne, Creteil (FR); Assistance Publique - Hopitaux de Paris, Paris (FR)

(72) Inventors: Dulce Papy-Garcia, Chilly Mazarin (FR); Alexandre Fifre, Champs Sur Marne (FR); Bruno Dubois, Paris (FR); Sandrine Chantepie-Laborde, Draveil (FR); Mohand Ouidir Ouidja, Le Kremlin Bicetre (FR); Francis Nyasse, Paris (FR); Patricia Albanese, Vitry Sur Seine (FR)

(73) Assignees: Universite Paris Est Creteil Val De Marne, Creteil (FR); Assistance Publique - Hopitaux de Paris, Paris (FR)

(21) Appl. No.: 16/085,279

(22) PCT Filed: Mar. 15, 2017
(86) PCT No.: PCT/EP2017/056152

§ 371 Date: Sep. 14, 2018

(30) Foreign Application Priority Data
Mar. 15, 2016 (EP) 16305285.5

(51) Int. Cl. G01N 33/68; G01N 33/573; C12N 5/0786; G01N 33/52

(52) U.S. Cl. CPC G01N 33/6896; G01N 33/573; G01N 33/526; C12N 5/0645; G01N 33/6842

(57) ABSTRACT

The present invention relates to a method of prognosis and/or diagnosis of Alzheimer's disease by determining the level and/or cellular distribution of heparan sulfates (HS) and/or heparan sulfate sulfotransferases (HSSTs) from isolating circulating immune cells in said circulating immune cells.

The present invention relates to a method of prognosis and/or diagnosis of Alzheimer's disease (AD) from isolated circulating immune cells by determining the level and/or cellular distribution of heparan sulfates (HS) and/or of heparan sulfate sulfotransferases (HSSTs) in said isolated circulating immune cells.

Neurodegenerative diseases, in particular AD, have a strongly debilitating impact on patient's life. Furthermore, these diseases constitute an enormous health, social, and economic burden. AD is the most widespread neurodegenerative disease globally and is estimated to afflict more than 27 million people worldwide. AD accounts for at least 50-70% of all dementia diagnosed clinically and it is probably the most devastating age-related neurodegenerative condition affecting about 10% of the population aver 65 years of age and up to 50% over age 85. The age of onset of AD may vary within a range of 50 years, with early-onset AD occurring in people younger than 65 years of age, and late-onset of AD occurring in those older than 65 years. About 10% of all cases suffer from early-onset AD, with only 1-2% being familial, mutant based, inherited cases, the remaining 98-99% are sporadic in where no mutations are associated to the disease.

Clinically, AD is a progressive disease that is associated with early deficits in memory formation and ultimately leads to the complete erosion of higher cognitive function. The cognitive disturbance includes, among other things, memory impairment, aphasia, agnosia and the loss of executive functioning. A characteristic feature of the pathogenesis of AD is the selective vulnerability of particular brain regions and subpopulations of nerve cells to the degenerative process. Specifically, the cortex temporal lobe region and the hippocampus are affected early and more severely during the progression of the disease. Currently, there is no cure for AD, nor is there an effective treatment to halt its progression. To date, AD prognosis and diagnosis from blood or plasma samples are still not available.

The biomarker research for AD has significantly advanced in recent years. The body fluids such as cerebrospinal fluid (CSF), plasma, and urine are considered as important sources for the AD biomarker development. To date, the most effective methods for establishing the diagnosis of AD are defined by multi-modal pathways, starting with clinical and neuropsychological assessment, CSF analysis, and brain-imaging procedures. At present, at least six biochemical measurements or scanning procedures have been validated and are used as biomarkers. These gold-standard biomarkers fall into two categories: a) biomarkers assessed by analysis of cerebrospinal fluid (CSF) for levels of amyloid B42 (AB42), total tau, and phosphorylated tau, and b) neuroimaging measures—hippocampal atrophy measured by magnetic resonance imaging (MRI), amyloid uptake as measured by Pittsburg compound B positron emission tomography (PiB-PET), or other F18 tracers, and decreased fluorodeoxyglucose (¹⁸F) uptake as measured by PET (FDG-PET) (Cavedo et al., 2014).

These markers have been validated by international expert working groups for their use on the diagnosis of AD dementia, mild cognitive impairment (MCI) due to AD, prodromal AD and preclinical AD. However, all these markers have no prognostic/diagnostic values and they are characterized by significant cost- and access-to-care barriers. Thus, several reviews on the current state-of-the-art on both biological and neuroimaging derived biomarker discovery focus on the necessity of development and validation of robust and reproducible non-invasive generalizable blood-based biomarkers with ideally diagnostic and prognostic values (Cavedo et al., 2014. Ritter et al., 2015). However, applicability of CSF in AD diagnosis is limited by lumbar puncture which, has the inconvenient to be a complex, expensive and invasive method. Moreover, it can lead to inconsistency of data analysis due to sample collection, transportation, storage, and limit its use as a routine diagnosis.

Blood-based measures of biomarkers provide a minimally invasive option for endo-phenotype earlier markers. Several approaches have considered the study of marker proteins in serum or plasma including AB, tau, and/or markers of inflammation. Others have proposed blood-based proteomic biomarkers that may have diagnostic utility in discriminating AD cases from control. However, a limited success has been reported in identifying a reproducible signature of diagnostic or trials utility.

Lipidomic or metabolomics signatures in plasma, serum or circulating cells approaches have shown to be promising for established AD, however they have failed to detected preclinical or prodromal disease (Baird et al., 2015).

The advent of new potent technological tools, including microarray technology, next generation sequencing transcriptome, epigenetic analysis, and detection of microRNAs (miRNAs) in peripheral biofluids such as plasma, serum, urine and cerebrospinal fluid as well as in mononuclear cells are expected to help in identifying new AD-markers, although today there is not proof their real value on AD prognosis or diagnosis (Bossù et al;, 2015; Mushtaq et al., 2015).

The value of myeloid cells including blood-borne monocytes, macrophages, and dendritic cells in AD has been proposed by performing flow cytometric analysis of defective Abeta phagocytosis by blood-derived monocytic cells that is altered in patients with AD, although not information exist for their AD-prognostic value (Fiala et al., 2010).

Unfortunately, to date none of the biomarkers presently available are able to accomplish the disease diagnosis single-handedly and monitoring more than one biomarker at the same time is suggested to be suitable for detecting the disease progression (Anoop et al, 2010).

Thus, there is a need for new biological markers for the prognosis and/or diagnosis AD which would be non-invasive, reliable, with a faster time to results, less expensive and that could be used as a routine diagnosis.

Therefore, one of the aims of the invention is to provide a method of prognosis and/or diagnosis of Alzheimer's disease based on the intracellular accumulation and distribution of HS and HSSTs in immune circulating cells.

One of the aims of the invention is to provide a method of prognosis and/or diagnosis of Alzheimer's disease from isolated circulating immune cells.

Another aim of the invention is to provide HS and/or HSSTs, preferably 3S-HS and/or HS3ST for use as a circulating biological marker for Alzheimer disease. Preferentially said HS3ST is selected from HS3ST1, HS3ST2, HS3ST3A, HS3ST3B, HS3ST4 and HS3ST5 and HS3ST6, preferably said HS3ST is selected from HS3ST2, HS3ST3A, HS3ST3B and HS3ST5.

According to the invention, said circulating biological markers for Alzheimer disease can be detected with specific antibodies, primer combinations or probes.

Another aim of the invention is to provide a kit for the implementation of said method of diagnosis or prognostic of Alzheimer's disease.

The present invention relates to an in vitro method of prognosis and/or diagnosis of Alzheimer's disease, in a subject comprising the steps of:
a) isolating circulating immune cells from said subject;
b) determining the level and/or cellular distribution of heparan sulfates (HS) and/or heparan sulfate sulfotransferases (HSSTs) in said circulating immune cells;
c) comparing:
   said level and/or cellular distribution of HS and/or HSSTs; or
   the ratio of said level of HSSTs to said level of HS,
to a respective reference representing a known disease or health status.

Advantageously, said ratio of said level of HSSTs to said level of HS of the step c) of the method according to the invention consists of the level of HS3ST2, HS3ST3A, HS3ST3B and HS3ST5, to the level of 3S-HS, preferentially the level of HS3ST3A or HS3ST3B to the level of 3S-HS, more preferentially the level of HS3ST3B to the level of 3S-HS.

In the present invention, the terms “risk”, “susceptibility”, and “predisposition” are tantamount and are used with respect to the probability of developing a neurodegenerative disease, preferably Alzheimer's disease. The term “AD” shall mean Alzheimer's disease. “AD-type neuropathology” as used herein refers to neuropathological, neurophysiological, histopathological and clinical hallmarks as described in the instant invention and as commonly known from state-of-the-art literature (Ballard et al, 2011).

By “subject” it is meant mammalian, preferably human, having or susceptible to have AD. Preferentially, said subject have or are susceptible to have familial AD or mutant based AD which is not associated with inherited mutation. In a particular embodiment, the term “subject” does not encompassed subject having a familial, mutant based, inherited AD.

The expression “heparan sulfate” (HS) refers to total heparan sulfate comprising heparan sulfate bearing N-, 2-O, and 6-O sulfates. The term “HS” shall mean Heparan sulfates independently of specific sulfation patterns. In particular, heparan sulfate bearing 3-O-sulfates also called 3-O-sulfated heparan sulfates (3S-HS), which can also be detected in circulating cells. The term “3S-HS” shall mean 3-O-sulfated heparan sulfates bearing at least a sulfate group in the position 3 of a glucosamine HS moiety in addition to other classic N-6-O and 2-O sulfations. 3S-HS are rare forms of HS resulting from specific pathways during HS biosynthesis. Structurally, HS chains are formed of a repeating disaccharide unit composed of an uronic acid linked to an N-acetyl glucosamine (GlcNAc). During biosynthesis, the elongating disaccharide chain follows several modifications including epimerization by a C5-epimerase transforming the uronic acid (GlcA) into iduronic acid (IdoA), and various region-selective sulfations assured by different sulfotransferases (Sandwall et al, 2010) including NDSTs (N-deacetyl-O-sulfotransferases), HS2ST (2-OST), HS6ST (6-OST) and HS3ST (3-OST), which respectively introduce sulfates groups at the 2-O-position of the IdoA, at the 6-O-position of GlcN or at the 3-O-position of the GlcN. A well-orchestrated expression of the various sulfotransferases results in a good cell controlled diversity of HS sequences.

The term “highly sulfated HS” is here defined as HS comprising disaccharide sequences having at least 3 of the following sulfated positions: N-sulphation, 2-O-, 6-O- and 3-O-sulphation. Highly sulfated HS disaccharides according to the invention includes: N-sulphate, 2-O-, 6-O- and 3-O-sulphate; N-sulphate, 2-O- and 6-O-sulphate; N-sulphate, 6-O- and 3-O-sulphate; N-sulphate, 2-O- and 3-O-sulphate; N-acetyl, 2-O-, 6-O- and 3-O-sulphate; N, 2-O, 6-O- and 3-O-sulphate and/or combination of oligosaccharides or polysaccharide HS chain containing at least one of these structures.

HS are well recognized to play important biological roles as regulators of the functions of a family of proteins known as heparin binding proteins (HBP), which include several growth factors, matrix proteins, cytokines, etc. The structures and regulatory activities of HS are basically exerted through specific sulfation of the HS chains at positions N-2-O, and 6-O. The 3-O-position has only been directly related to anticoagulation and virus infection. Interestingly, these HS structures are highly constant in tissues but vary from one tissue to another to appropriately fit each tissue function. Observations in brains from AD and Down syndrome (DS) have shown that HS accumulates at the intracellular levels in neurons of affected subjects but not in those from control individuals (Snow et al., 1990; Goedert et al., 1996). Interestingly, in DS brain, the intracellular accumulation of HS precedes from several years the detection of amyloid plaques and neurofibrillary tangles (NFTs) (Snow et al., 1990).

Moreover, according to observations in brain from young subjects having DS, which develop AD in adulthood, neuronal cell presenting HS intracellular accumulation are observed and should be detectable decades before the disease onset (Su et al., 1996), as well as in early and in advanced AD.

Accordingly, in a particular embodiment, said step b) of the in vitro method of prognosis and/or diagnosis of AD can also consist in determining cell size and/or morphology of said circulating immune cells, and wherein said step c) consist in comparing said cell size and/or morphology of circulating immune cells containing said HS and/or HSSTs. For example, said cell size and/or morphology (i.e. granulometry . . . ) of cells containing said HS and/or HSSTs can present an increase of their size average of about 120-200%, preferably 150% compared to the size of said circulating immune cells in a healthy patient. Advantageously, said step c) of comparing cell morphology is used for prognostic or diagnosis of AD.

Preferentially, the in vitro method of prognosis and/or diagnosis of Alzheimer's disease according to the invention, determine in step b) the level and/or cellular distribution of HS and/or HSSTs, wherein said HS is 3-O-sulfated heparan sulfates (3S-HS), and said HSSTs are selected from heparan sulfate 3-O-sulfotransferases (HS3ST), preferentially HS3ST1, HS3ST2, HS3ST3A, HS3ST3B, HS3ST4, HS3ST5 and HS3ST6, more preferentially HS3ST2, HS3ST3A, HS3ST3B and HS3ST5.

The terms heparin-glucosamine 3-O-sulfotransferase-n or heparan sulfate (glucosamine) 3-O-sulfotransferase-n, with ‘n’ being 2 to 6, have the same meaning. These enzymes can also be named 3-OST-n or 3OSTn, HS3STn, h3-OSTn; heparan sulfate 3-O-sulfotransferase-n; heparan sulfate D-glucosaminyl 3-O-sulfotransferase-n; heparan sulfate glucosamine 3-O-sulfotransferase-n.

The term “variant” as used herein refers to any polypeptide or protein, in reference to polypeptides and proteins disclosed in the present invention, in which one or more amino acids are added and/or within the native amino acid sequences of the native polypeptides or proteins of the present invention. Furthermore, the term “variant” shall include any shorter or longer version of a polypeptide or protein, “variants” shall also comprise a sequence that has at least 80% sequence identity, more preferably at least 90% sequence identity, more preferably at least 95% sequence identity with the amino acid sequence of HS3ST1 (SEQ ID NO. 1; Accession number: AAH57803.1), HS3ST2 (SEQ ID NO. 2; Accession number: AAQ89453.1), HS3ST3A (SEQ ID NO. 3; Accession number: Q9Y663.1), HS3ST3B (SEQ ID NO. 4; Accession number: Q9Y662), HS3ST4 (SEQ ID NO. 5; Accession number: AAD30210.2), HS3ST5 (SEQ ID NO. 6; Accession number: EAW48251.1) and HS3ST6 (SEQ ID NO. 7; Accession number: NP_001009606). “Variants” of a protein molecule include, for example, proteins with conservative amino acids substitutions in highly conservative regions. “Proteins and polypeptides” of the present invention includes variants, fragments and chemicals derivatives of the protein comprising the amino acid sequence of HS3ST1 (SEQ ID NO. 1), HS3ST2 (SEQ ID NO. 2), HS3ST3A (SEQ ID NO. 3), HS3ST3B (SEQ ID NO. 4), HS3ST4 (SEQ ID NO. 5), HS3ST5 (SEQ ID NO. 6) and HS3ST6 (SEQ ID NO. 7). Sequences variations shall be included wherein a codon is replaced with another codon due to alternative base sequences, but the amino acid sequence translated by the DNA sequence remains unchanged. This known in the art phenomenon is called redundancy of the set of codons which translate specific amino acids. Included shall be such exchange of amino acids which would have no effect on the functionality, such as arginine for lysine, valine for leucine, asparagine for glutamine. Proteins and polypeptides can be included which can be isolated from nature or be produced by recombinant and/or synthetic means. Native proteins or polypeptides refer to naturally-occurring truncated or secreted forms, naturally occurring forms (e.g. splice-variants and naturally occurring allelic variants).

The terms “circulating immune cells” as used herein refers to peripheral blood mononuclear cells (PBMC) selected from T cells, B cells, monocytes and/or monocyte derived macrophages (MDM), polymorphonuclear cells (PMNs) as well as dendritic cells (DCs).

In a preferred embodiment, in step a) of the in vitro method of prognosis and/or diagnosis of Alzheimer's disease according to the invention, said circulating immune cells from a subject are selected from circulating T cells, B cells, monocytes and/or MDM, preferentially circulating monocytes and/or MDM.

Moreover, it has been observed that the level and/or cellular distribution of HS and/or HSSTs would be enhanced when said circulating immune cells were cultured during several days. These level and/or cellular distribution of HS and/or HSSTs could further be enhanced when said monocytes and/or MDM were cultured and differentiated in M0 macrophage phenotype, and even further when differentiated in M1 and M2 macrophage phenotypes.

In a preferred embodiment, the in vitro method of prognosis and/or diagnosis of Alzheimer's disease according to the invention, further comprises after said step a) a culturing step a1) of said circulating immune cells, preferably monocytes and/or MDM, in an appropriate culture medium, such as RPMI 1640 medium. Advantageously, said culturing step a1) is performed between 7-10 days, preferably 10 days, within an appropriate culture medium according to the invention complemented with Macrophage Colony-Stimulating Factor (M-CSF) until said monocytes and/or MDM present a M0 phenotype. Preferably, the in vitro method of prognosis and/or diagnosis of Alzheimer's disease according to the invention, wherein said circulating immune cells are selected from monocytes and/or MDM, and wherein said culturing step a1) is performed between 7-10 days, preferably 10 days, within an appropriate culture medium according to the invention complemented with Macrophage Colony-Stimulating Factor (M-CSF) until said monocytes and/or MDM present a M0 phenotype.

By “appropriate culture medium” it is meant any cell culture medium suitable for circulating immune cells, such medium is known by the skilled person and are for example RPMI 1640 medium supplemented or not with 10% male AB human serum (HuS) and Penicillin/streptavidin (P/S).

The term “M0 phenotype” refers to monocyte derived naive macrophages, CD16+ and/or CD16−, that have not yet differentiated into either M1 or M2 macrophages, characterized by marker profile CD14⁺/CD68⁺/CD80⁻/CD163⁻/CD209⁻/CD206⁻ (Mantovani et al, 2012). Appropriate culturing medium to obtain M0 macrophage phenotype are for example cell culture media containing the bioactive protein M-CSF.

By “culturing step” it is meant herein that isolated cells are grown ex vivo and maintained in an appropriate culture medium at an appropriate temperature and gas mixture in a cell incubator. It is well known from the skilled person that culture conditions vary widely for each cell type, and those of skill in the art will thus recognize the appropriate culture conditions for a particular cell type. For example, said appropriate culture medium is a RPMI 1640 medium, containing 10% human serum (HuS, preference male).

In a preferred embodiment, the in vitro method of prognosis and/or diagnosis of Alzheimer's disease according to the invention, wherein said culturing step, following said step a1), comprises an additional culturing step a2) of 2 to 3 days, preferably 3 days, in an appropriate culturing cell medium according to the invention comprising IL4/IL10 until said M0 macrophage presents a M2 macrophage phenotype, or comprising pro-inflammatory factors, preferably Toll-like receptor (TLR) ligands, LPS and IFNγ, or other, until said M0 macrophage presents a M1 macrophage phenotype.

The term “M1 phenotype” or “M1 macrophages” is used throughout to refer to the subtype of macrophages activated by pro-inflammatory factors such as bacterial lipopolysaccharide (LPS) and interferon-γ (IFN-y) and demonstrating characteristics which include production of large amounts of pro-inflammatory signaling and effector molecules such as TNFα. A commonly accepted marker profile for M1 macrophages is CD14⁺/CD68⁺/CD80⁺/CD163⁻/CD209⁻/CD206⁻ (Mantovani et al, 2012). The main role of M1 cells is pathogen elimination and tissue destruction. Appropriate culturing medium to obtain M1 macrophage phenotype are for example RPMI 1640 medium, containing or not 10% human serum (HuS, male) and M-CSF complemented with pro-inflammatory factors, preferably Toll-like receptor (TLR) ligands, LPS and IFNγ.

The term “M2 phenotype” or “M2 macrophages” is used throughout to refer to the subtype of macrophages which are activated by anti-inflammatory factors, preferably interleukin-4 (IL-4), IL-10, IL-13, or a combination thereof and marker profile for M2 macrophages is CD14⁺/CD68⁺/CD80⁻/CD163⁺/CD209⁺/CD206⁺ many subtypes of M2 phenotype are covered by this term, e.g. M2a activated by IL-4/IL-13, M2b activated by immune complexes and M2c activated by IL-10. They would be known to a skilled person (e.g. described in Mantovani et al., 2004, 2012). These cells synthesize large amounts of anti-inflammatory IL10, TGF, and IL1 receptor antagonist, thus opposing the pro-inflammatory effects of M1 macrophages. Therefore, the main role of M2 cells is curbing inflammatory signaling, allowing resolution of inflammation, and tissue healing. Appropriate culturing medium to obtain M2 macrophage phenotype are for example RPMI 1640 medium, containing 10% human serum (HuS, male) and M-CSF complemented with anti-inflammatory factors such as recombinant human cytokines (IL4/IL10).

However, the skilled person will recognize appropriate culture conditions to apply to particular isolated circulating immune cells to differentiate them in M0, M1 or M2 macrophage phenotype.

Thus, in a preferred embodiment, the present invention also relates to an in vitro method of prognosis and/or diagnosis of Alzheimer's disease, in a subject comprising the steps of:
a) isolating monocytes and/or MDM from said subject;
a1) culturing said monocytes and/or MDM in an appropriate culture medium comprising M-CSF until said monocytes and/or MDM present a M0 macrophage phenotype;
b) determining the level and/or cellular distribution of 3S-HS and/or HS3ST in said circulating immune cells;
c) comparing:
   said level and/or cellular distribution of 3S-HS and/or HS3ST; or
   the ratio of said level of HS3ST to said level of 3S-HS,
   to a respective reference representing a known disease or health status.

And, in a preferred embodiment, the present invention relates to an in vitro method of prognosis and/or diagnosis of Alzheimer's disease, in a subject comprising the steps of:
a. isolating monocytes and/or MDM from said subject;
a1) culturing said monocytes and/or MDM in an appropriate culture medium comprising M-CSF until said monocytes and/or MDM present a M0 macrophage phenotype;
a2) culturing said M0 macrophage in an appropriate culture medium comprising IL4/IL10 until said M0 macrophage presents a M2 macrophage phenotype;
b. determining the level and/or cellular distribution of 3S-HS and/or HS3ST in said circulating immune cells;
c. comparing:
   said level and/or cellular distribution of 3S-HS and/or HS3ST; or
   the ratio of said level of HS3ST to said level of 3S-HS,
   to a respective reference representing a known disease or health status.

In a particular embodiment, said step b) of the in vitro method of prognosis and/or diagnosis of AD can also consist in determining cell size and/or morphology of said isolated monocytes and/or MDM, and wherein said step c) consist in comparing said cell size and/or morphology of cells containing said 3S-HS and/or HS3ST. For example, said cell size and/or morphology (i.e. granulometry . . . ) of cells containing said 3S-HS and/or HS3ST can present, like in DS, an increase of their size average of about 120-200%, preferably 150% compared to the size of said circulating immune cells in a healthy patient. Advantageously, said step c) of comparing cell morphology is used for early prognostic or diagnosis of AD.

In a particular embodiment, the in vitro method of prognosis and/or diagnosis of Alzheimer's disease according to the invention, determines in step b) the level of HS and/or HSSTs, by a method selected from immunofluorescence, Western Blot, ELISA, mass spectrometry, flow cytometry methods, immunohistochemistry methods, and combinations thereof.

In a particular embodiment, the in vitro method of prognosis and/or diagnosis of Alzheimer's disease in a subject according to the invention determines that said subject has said Alzheimer's disease when the comparing step c) shows that said level and/or said cellular location and/or distribution of HS3ST is altered.

In a particular embodiment, the in vitro method of prognosis and/or diagnosis of Alzheimer's disease in a subject according to the invention determines that said subject has said Alzheimer's disease when the comparing step c) shows that said level and/or said cellular morphology is altered.

By the term “altered” it is meant that the amount and cell location of HS3ST in the cell may be either subject to an increase or a decrease compared to a respective reference representing a known health status.

In a particular embodiment, the in vitro method of prognosis and/or diagnosis according to the invention shows that a subject has Alzheimer's disease when the comparing step c) concludes that said level of HS or 3S-HS is increased and/or its cellular location is altered, to be accumulated essentially into the cytosol but also in the nucleus.

Another object of the present invention relates to a circulating biological marker for Alzheimer's disease consisting of at least one of HS and/or HSSTs, preferentially at least one of 3S-HS and/or HS3ST. According to a preferred embodiment, said HS3ST is selected from HS3ST1, HS3ST2, HS3ST3A, HS3ST3B, HS3ST4, HS3ST5 and/or HS3ST6, preferentially from HS3ST2, HS3ST3A, HS3ST3B and/or HS3ST5.

Another object of the present invention is directed to a kit for the prognosis and/or diagnosis of Alzheimer's disease comprising purification means of circulating immune cells, preferably monocytes and/or monocytes derived macrophages (MDM), and detection means of level and/or cellular distribution of said HS and/or HSSTs, preferably 3S-HS and/or HS3ST, said HS3ST being preferentially selected from HS3ST1, HS3ST2, HS3ST3A, HS3ST3B, HS3ST4, HS3ST5 and/or HS3ST6, preferably HS3ST2, HS3ST3A, HS3ST3B and/or HS3ST5.

By “purification means” we refer herein to any mean suitable to isolate from a biological fluid sample circulating immune cells. Such means are for examples filters able to selectively retain or enrich in cells of sizes superior to 20 μm, preferably superior to 15 μm of diameter.

By “detection means” we refer herein to antibody, probe or primers, marked with a detectable marker, that are capable of identify cell size and/or specifically binding HS and/HSSTs of interest. Said detectable markers can be selected from colorimetric or fluorescent label, such as fluorochromes selected from PE-Cy5.5, PE-CF594, PE-Cy7, PE or FITC. Preferentially, specific markers of HS and HSSTs are marked with different colorimetric or fluorescent label, and more preferentially each of the specific markers used are marked with different colorimetric or fluorescent label. Advantageously, specific markers of HS and HSSTs are marked with different fluorochromes, and more preferentially each of the specific markers used are marked with different fluorochromes.

In a particular embodiment, the kit for the prognosis and/or diagnosis of Alzheimer's disease according to the invention further comprises:
at least one primer combination to amplify said HS and/or HSSTs, preferably 3S-HS and/or HS3ST, said HS3ST being preferentially selected from HS3ST1, HS3ST2, HS3ST3A, HS3ST3B, HS3ST4, HS3ST5 and/or HS3ST6, preferably HS3ST2, HS3ST3A, HS3ST3B and/or HS3ST5; and/or
at least one probe, such as nucleic acid probes, to detect said HS and/or HSSTs, preferably 3S-HS and/or HS3ST, said HS3ST being preferentially selected from HS3ST1, HS3ST2, HS3ST3A, HS3ST3B, HS3ST4, HS3ST5 and/or HS3ST6, preferably HS3ST2, HS3ST3A, HS3ST3B and/or HS3ST5; and/or
at least one specific antibody of said HS and/or HSSTs, preferably highly sulfated HS or HS chains bearing 3-O-sulfation (3S-HS), and/or HS3ST, said HS3ST being preferentially selected from HS3ST1, HS3ST2, HS3ST3A, HS3ST3B, HS3ST4, HS3ST5 and/or HS3ST6, preferably HS3ST2, HS3ST3A, HS3ST3B and/or HS3ST5; and/or

Advantageously, the kit for the prognosis and/or diagnosis of Alzheimer's disease according to the invention further comprises a notice of use.

The term “probe” refers to mixture of nucleic acids that are detectably labeled, e.g., fluorescently labeled, such that the presence of the probe, as well as, any target sequence to which the probe is bound can be detected by assessing the presence of the label. The term “nucleic acid” and “polynucleotide” are used interchangeably herein to describe a polymer of any length composed of nucleotides, e.g., deoxyribonucleotides or ribonucleotides, or compounds produced synthetically which can hybridize with naturally occurring nucleic acids in a sequence specific manner analogous to that of two naturally occurring nucleic acids, e.g., can participate in Watson-Crick base pairing interactions.

In a preferred embodiment, the kit according to the invention comprise specific conjugated antibody linked to a colorimetric or fluorescent label.

In a preferred embodiment, the kit according to the invention comprises purification means consisting of filters which are selectively retaining circulating immune cells, preferably circulating monocytes and/or MDM.

A kit according to the invention, wherein said kit further comprises:
buffers, preferably Phosphate Buffered Saline;
an appropriate cell culture medium according to the invention (such as a serum);
M-CSF;
optionally pro and/or anti-inflammatory factors, preferably selected from recombinant human cytokines IL4 and IL10; or Toll-like receptor (TLR) ligands, LPS and IFNγ; and
one or more cell culture containers.

The term “buffer” refers to a solution suitable for cell culture, comprising in particular sodium phosphate, which are commonly used by the person skilled in the art. Examples of such buffer are Phosphate Buffered Saline.

The present invention is also related to a method of treatment of Alzheimer's disease in a subject in need thereof comprising the steps of:
a) isolating circulating cells from circulating immune cells, preferably circulating monocytes and/or MDM of said subject,
b) optionally culturing said circulating monocytes and/or MDM within an appropriate culture medium,
c) determining the level and/or cellular distribution of said HS and/or HSSTs, preferably 3S-HS and/or HS3ST, said HS3ST being preferentially selected from HS3ST1, HS3ST2, HS3ST3A, HS3ST3B, HS3ST4, HS3ST5 and/or HS3ST6, preferably HS3ST2, HS3ST3A, HS3ST3B and/or HS3ST5;
d) comparing:
   said level and/or cellular distribution of HS and/or HSSTs; or
   the ratio of said level of HSSTs to said level of HS,
   to a respective reference representing a known disease or health status;
e) selecting subject presenting abnormal level and/or cellular distribution, or ratio, of said HS and/or HSST according to step d),
f) administering to said selected subject of step e) at least one agent suitable for treating Alzheimer's disease.

In a particular embodiment, said step c) of the in vitro method of treatment of AD can also consist in determining cell size and/or morphology of said circulating immune cells, and wherein said step d) consist in comparing said cell size and/or morphology (i.e. granulometry . . . ) of cells containing said 3S-HS and/or HS3ST. For example, said cell size of cells containing said highly sulfated HS and/or 3S-HS and/or HS3ST can present, like in DS, an increase of their size average of about 120-200%, preferably 150% compared to the size of said circulating immune cells of a healthy patient.

By “agent suitable for treating Alzheimer's disease” it is meant any suitable agent susceptible to be administered to a patient suffering, or susceptible to suffer from AD, such said at least one agent suitable for treating Alzheimer's disease can for example be selected from antibody-based therapies (BIIB037, Aducanumab, Crenezumab, Ponezumab, Bapineuzumab, etc.), vacins (AFFITOPE, CAD106, ACC-001, etc.), serotoninergic agents (Dimebon, Pimavanserin, Cerlapirdine, Citalopram, etc.), Cholinergic agents (Donepezil, Rivastugmine, Encenicline, rivastigmine, etc.), other neurotransmitters and their receptors interacting agents (Memantine, Acamprosate, Sifrol, Brexpiprazole, PXT00864, etc.), BACE inhibitors (MK-8931, E2609, etc.), Gamme secretase inhibitors (Begacestad, Semagacestat, etc.), kinase inhibitors (Tideglusib, Bryotatin, etc.), anti-epileptics/antipsychotics (Quetiapine, NSA-789, etc.), anti-inflammatory agents (AZD5213, GC021109, indomethacine, etc), diet supplements (NeuroAD, L-Arginine, Simvastatin, tetrahydrobiopterin, vitamins, etc.), cell therapy agents (Neurostem, etc.), brain stimulation interventions (Rtms), anti-tau drugs (TRx0237, etc.), anti-tau antibodies, anti-tau oligonucleotides among others.

Another object of the present invention is directed to an in vitro method for measuring level and/or cellular distribution of HS and HSSTs, preferably highly sulfated HS and 3S-HS and/or HS3ST selected from HS3ST1, HS3ST2, HS3ST3A, HS3ST3B, HS3ST4, HS3ST5 and HS3ST6 in circulating immune cells of a subject, said method comprising the steps of:
a) isolating circulating immune cells, preferably circulating monocytes and/or MDM of said subject,
b) optionally culturing said circulating immune cells within an appropriate culture medium to obtain M0, M1 or M2 macrophage phenotype,
c) determining the level and/or cellular distribution of said HS and/or HSSTs, preferably 3S-HS and/or HS3ST, said HS3ST being preferentially selected from HS3ST1, HS3ST2, HS3ST3A, HS3ST3B, HS3ST4, HS3ST5 and/or HS3ST6, preferably HS3ST2, HS3ST3A, HS3ST3B and/or HS3ST5,
d) comparing:
   said level and/or cellular distribution of HS and/or HSSTs of step c); or
   the ratio of said level of HSSTs to said level of HS of step c),
   to a respective reference representing a known disease or health status.
   wherein said subject is suspected to suffer from Alzheimer's disease or is suffering from Alzheimer's disease.

In a particular embodiment, said step c) of the in vitro method for measuring level and/or cellular distribution of HS and HSSTs, preferably 3S-HS and/or HS3ST selected from HS3ST1, HS3ST2, HS3ST3A, HS3ST3B, HS3ST4, HS3ST5 and HS3ST6 in circulating immune cells can also consist in determining cell size and/or morphology of said circulating immune cells, and wherein said step d) consist in comparing the cell size and/or morphology of cells containing said HS and/or HSSTs of step c).

Still another object of the present invention is directed to an in vitro method for determining whether a subject is at risk of developing Alzheimer's disease, comprising the steps of:
a. isolating circulating immune cells, preferably circulating monocytes and/or MDM of said subject,
b. culturing said circulating immune cells within an appropriate culture medium to obtain M0, M1 or M2 macrophage phenotype,
c. determining the level and/or cellular distribution of HS and/or HSSTs, preferably 3S-HS and/or HS3ST, said HS3ST being preferentially selected from HS3ST1, HS3ST2, HS3ST3A, HS3ST3B, HS3ST4, HS3ST5 and/or HS3ST6, preferably HS3ST2, HS3ST3A, HS3ST3B and/or HS3ST5,
d. comparing:
   said level and/or cellular distribution of HS and/or HSSTs; or
   the ratio of said level of HSSTs to said level of HS,
   to a respective reference representing a known disease or health status, and
   wherein said subject is susceptible of developing Alzheimer's disease or is suffering from Alzheimer's disease.

In a particular embodiment, said step c) of the in vitro method for determining whether a subject is at risk of developing Alzheimer's disease can also consist in determining cell size and/or morphology of said circulating immune cells, and wherein said step d) consist in comparing said cell size and/or morphology of cells containing said HS and/or HSSTs of step c).

The foregoing are merely examples of some of embodiments of the methods, and kits of the invention can be used, and are not intended to be limiting.

### FIGURES

The invention will be further described and illustrated with reference to the accompanying drawings in which:

FIG. 1. Method of isolating Monocytes (CD14+ cells) from a blood sample. PMBC are isolated from EDTA blood samples by methods classically used by the men of the art, for instance Ficoll devises, magnetic beads or cell selecting filters. Monocytes are further isolated from PBMC by strategies classically used by the men of the art, for instance using CD14-magnetic beads. Obtained monocytes (CD14+) can be used for direct analysis or for primary culture.

FIG. 2. Method of cultured isolated monocytes (CD14+) and differentiation to obtain M0, M1 or M2 macrophage phenotypes. Culture conditions are as those used by the men of the art. For instance, monocytes are cultured in RPMI 1640 containing 10% HuS. Adherent cells are then cultured in the presence of M-CSF to obtain M0 phenotype. M1 phenotype is obtained by including pro-inflammatory factors, for instance IFNγ and LPS, in the cell medium of M0 cultured cells. M2 phenotype is obtained by including anti-inflammatory factors, for instance IL4 and IL10, in the cell medium of M0 cultured cells. Compounds concentrations and culturing times are as those showed in the figures.

FIG. 3. Accumulation and cellular distribution of 3S-HS and HS3ST2 in MDM M0 from Patient 1 and Control individual 1. Differential cellular accumulation and distribution of 3S-HS and HS3ST2 immunostaining in MDM M0 from an AD patient vs control individual. Cell nuclei is shown by DAPI staining. A) Confocal optical slide at a z plane near the cell basement, B) confocal optical slide at a z plane at the middle cell level, C) total confocal projection of the entire cell, and D) phase contrast.

FIG. 4. Accumulation and cellular distribution of 3S-HS and HS3ST2 in MDM M0 from AD Patient and Control individual. Differential cellular accumulation and distribution of 3S-HS and HS3ST2 immunostaining in MDM M0 from AD vs control individuals. Cell nuclei is shown by DAPI staining. A) Confocal optical slide at a z plane near the cell basement, B) confocal optical slide at a z plane at the middle cell level, C) total confocal projection of the entire cell, and D) phase contrast.

FIG. 5. Accumulation of 3S-HS in MDM M0 from Alzheimer's disease vs inflammatory disease Osteoarthritis (OA). The figure shows 5 Alzheimer's disease (AD) patients, 5 control individuals and 5 Osteoarthritis (OA) patients. Total projections of the MDM M0 cells from AD cells shows characteristic AD immunostaining

### EXAMPLES

### Material and Methods

### Isolation of Circulating Immune Cells

Peripheral Blood Mononuclear Cells (PBMC) isolation. K2-EDTA venous blood samples are used to isolate PBMC. Ficoll method is used for separating and isolation PBMC—most specifically lymphocytes and monocytes. Blood specimens are carefully layered on top of the Ficoll-Paque Plus solution, and then briefly centrifuged to form different layers containing different types of cells. The bottom layer is made up of red blood cells (erythrocytes) which are collected or aggregated by the Ficoll medium and sink completely through to the bottom. The next layer up from the bottom is primarily granulocytes, which also migrate down through the Ficoll-Paque Plus solution. The next layer toward to top, which is typically at the interface between the plasma and the Ficoll solution, is the lymphocytes along with monocytes and platelets. To recover these cells, Ficoll-Paque Plus fabricant instructions are followed, this is largely known by men or the art.

Lymphocytes and monocytes independent isolation and characterization. After Ficoll isolation of PBMC, lymphocytes and monocytes are independently isolated by using immune-magnetic beads (Myltenyi MACS microbeads) on the basis of surface markers selections with monoclonal specific antibodies. T cells are separated by using CD3+ coated beads. B cells are separated by using CD19+ coated beads. Monocytes are separated by using CD14+ coated beads. This technique, well known by the skill person, allows the separation of cells in relatively short time. Cells purity is assessed by FACScan analysis with the specific antibodies as classically performed by the skilled person. T cells are CD3+, B cells are CD19+, monocytes are CD14+ and CD68+. Cell purity is typically of at least 95% when assessed by flow cytometry. Each experiment is conducted with cells isolated from a single donor, in any case cells from different donors are combined.

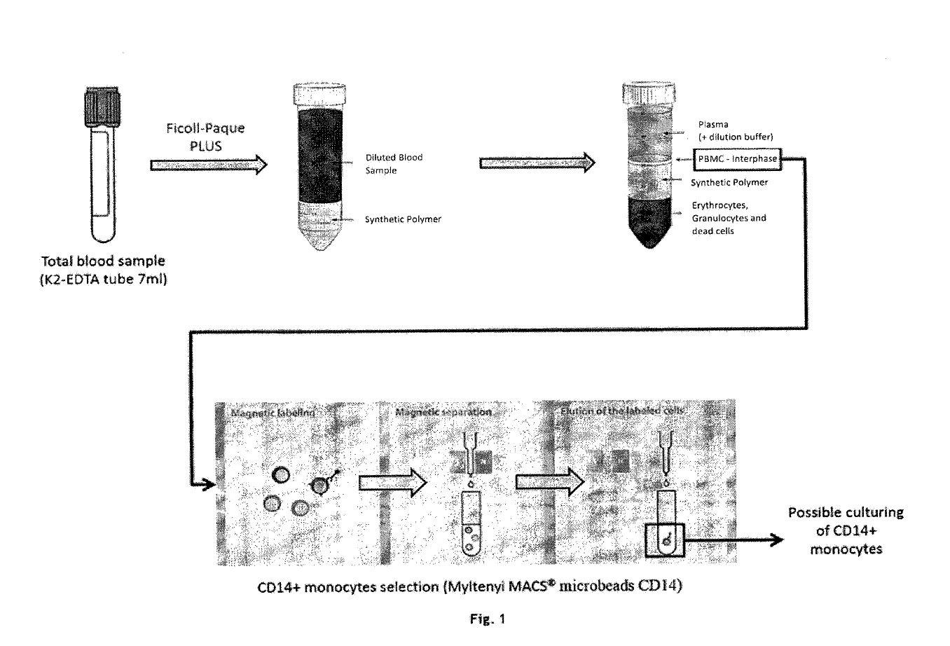

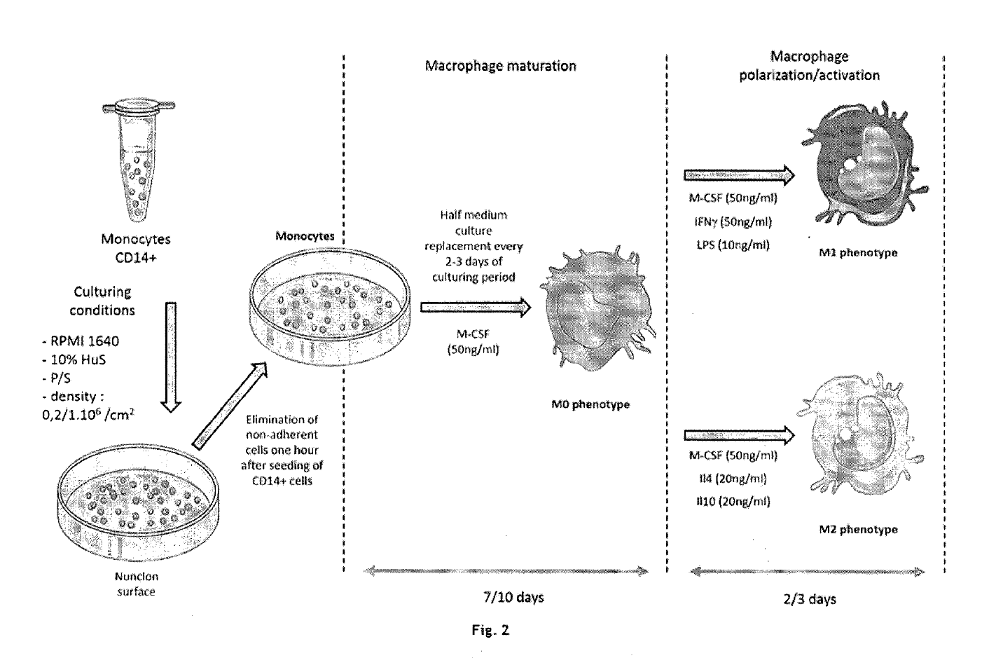

### Culture and Differentiation of Macrophage to M0, M1 and M2 Phenotype (FIG. 1)

MDM phenotypes M0, M1 and M2 induction. Freshly isolated monocytes (CD14+ cells) are cultured in complete “RPMI 1640 medium (Gibco 21875-034)” containing 10% human serum (HuS, male), classically at 0.2/1×10⁶ cells/cm². Cells are stimulated for instance during 7-10 days with M-CSF (for instance 50 ng/mL) to induce MDM to M0 phenotype; then cells are further stimulated for instance during 2-3 days with M-CSF (for instance 50 ng/mL) with inflammatory cytokines or with Toll-like receptor (TLR) ligands (for instance LPS 10 ng/mL; IFNγ 50 ng/mL) to induce the polarization of M0 macrophage into M1 phenotype or with M-CSF (for instance 50 ng/mL) and recombinant human anti-inflammatory cytokines (for instance IL4 20 ng/mL; IL10 20 ng/mL) to induce the polarization of M0 macrophage into M2 phenotype. MDM M0 is characterized by flow cytometry as CD14+, CD68+, CD80−, CD163−, CD206−, and CD209−. MDM M1 is characterized by flow cytometry as CD14+, CD68+, CD80+, CD163−, CD206−, and CD209−. MDM M2 is characterized by flow cytometry as CD14+, CD68+, CD80−, CD163+, CD206+, and CD209+.

Immunofluorescence. Cultured PBMC whatever the phenotype is (T cells, B cells, monocytes, or MDM M1, M2, or M0) are separately labelled with specific antibodies. Heparan sulfates in cells are labelled with HS-recognizing antibody HS4C3 (3S-HS), which is a phage display antibody able to selectively detect HS-3S, or by any other antibodies able to selectively detect HS having 3-O-sulfates), or by any other anti-HS antibody, and by 3-O-sulfotransferases (HS3ST1, HS3ST2, HS3ST3A, HS3ST3B, HS3ST4, HS3ST5, or HS3ST6) recognizing antibodies. Labelling is revealed as classically do by the skilled person by using the secondary and tertiary antibodies coupled to fluorescent probes (Alexa-Fluor probes are here classically used). The phage display HS4C3 antibody was revealed by an anti-VSV antibody and the by a fluorescent tertiary antibody.

Cell morphology, HS and HS3ST levels and cellular localization are assessed by microscopy, flow cytometry, fluorescence microscopy and/or by confocal fluorescence microscopy.

Other detection techniques such as Western blot, ELISA or other immunologic techniques can also be used.

### Example 1: HS3ST2 and HS-3S Immunolabelling in M2 MDM from AD and Control Individuals

Blood was collected from a control and an AD subject. AD patient was clinically characterized and previously diagnosed to be AD by amyloid uptake as measured by Pittsburg compound B positron emission tomography (PiB-PET) as do the men of the art. PBMC were isolated as described in materials and methods as do the men of the art. Isolated PBMC were used to isolate monocytes (CD14⁺ cells) that were then cultured in complete RPMI 1640 medium (Gibco 21875-034) containing 10% HuS (male) at 0.2/1×10⁶ cells/cm². Cells were then stimulated during 10 days with M-CSF (50 ng/mL) to induce MDM to M0 phenotype. M0 MDM were characterized by flow cytometry with next antibodies CD14+, CD68+, CD80−, CD163−, CD206−, and CD209−, as classically do the men of the art. Cultured MDM M0 were then labelled with specific primary antibodies against HS3ST2 and HS4C3. HS3ST2 labelling was revealed as classically do a skilled person by using the secondary antibody labelled with Alexa-Fluor 555. The phage display HS4C3 antibody was revealed by an anti-VSV antibody and then by a tertiary antibody labelled with Alexa-Fluor 488. Stack images were obtained with the software CellSens from a spinning disk inverted confocal microscope (IX81 DSU Olympus, 60N.A. 1.35) coupled to an Orca Hamamatsu RCCD camera. Images were processed with the ImageJ software (W. Rasband, National Institute of Health).

### Results

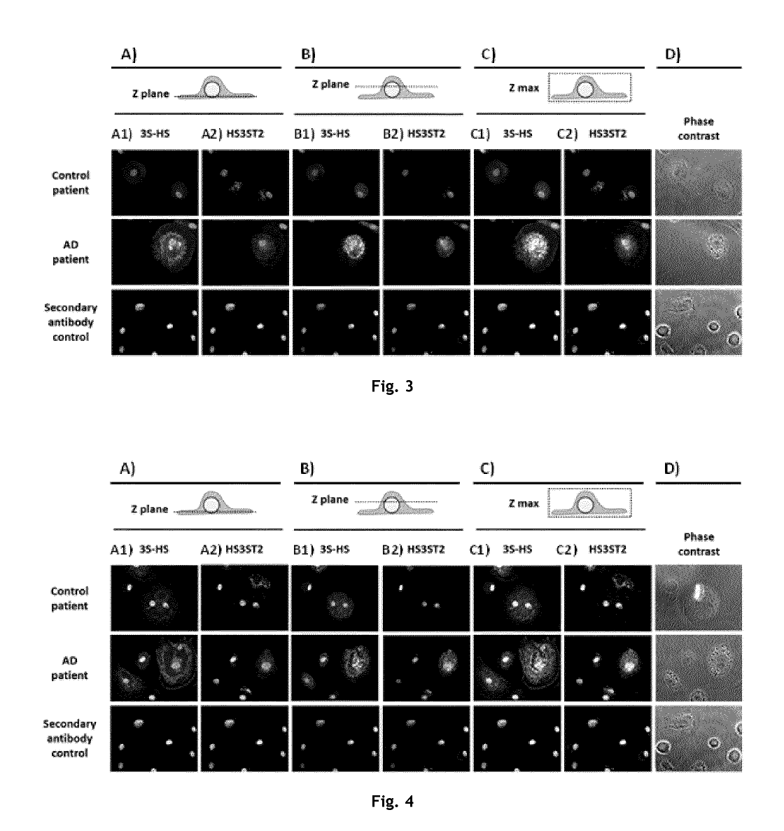

FIG. 3 shows the altered cellular accumulation and altered distribution of 3S-HS and HS3ST2 in the cell membrane and at the intracellular level of MDM M0 from AD vs control individuals. 3S-HS enhanced immunostaining in AD patient is observed in both confocal optical slides at a z axis plane near the cell basement (FIG. 3A1), at the middle of the cell (FIG. 3B1), and reflected by the total confocal projection of the entire cell (FIG. 3C1). HS3ST2 immunostaining shows altered cellular accumulation, while in control cells HS3ST2 accumulates in the ruffling regions near the cell basement (FIG. 3A2), in AD HS3ST2 acquired a diffuse cytosolic distribution (FIG. 3B2). This intracellular cellular relocation 3S-HS is also observed in the total confocal projection of the entire cell (FIG. 3C2).

### Conclusion

Compared to control MDM M0, AD shows morphologically altered MDM M0, which accumulate 3S-HS at the intracellular level and at the cell membrane. This is accompanied by an altered cell distribution of HS3ST2, which disappears from the ruffing areas to be located at the cytosol in the AD cells. In conclusion, the differential 3S-HS levels and distribution observed in AD can be used to determine whether a patient is affected or not by AD.

### Example 2: HS3ST2 and HS-3S Immunolabelling in M0 MDM from AD and Control Individuals

Blood was collected from a second control and a second AD subject. AD patient was clinically characterized and previously diagnosed to be AD by amyloid uptake as measured by Pittsburg compound B positron emission tomography (PiB-PET) as do the men of the art. PBMC were isolated as described in materials and methods as do the men of the art. Isolated PBMC were used to isolate monocytes (CD14⁺ cells) that were then cultured at 1×10⁶ per mL in complete RPMI 1640 medium (Gibco 21875-034) containing 10% HuS (male) at 0.2/1×10⁶ cells/cm². Cells were then stimulated during 7 days with M-CSF (50 ng/mL) to induce MDM to M0 phenotype. M0 MDM were characterized by flow cytometry with next antibodies CD14+, CD68+, CD80−, CD163−, CD206−, and CD209−, as classically do the men of the art. Cultured MDM M0 were then labelled with primary antibodies HS3ST2 and HS4C3. HS3ST2 labelling was revealed as classically do a skilled person by using the secondary antibody labelled with Alexa-Fluor 555. The phage display HS4C3 antibody was revealed by an anti-VSV antibody and then by a tertiary antibody labelled with Alexa-Fluor 488. Stack images were obtained with the software CellSens from a spinning disk inverted confocal microscope (IX81 DSU Olympus, 60N.A. 1.35) coupled to an Orca Hamamatsu RCCD camera. Images were processed with the ImageJ software (W. Rasband, National Institute of Health).

### Results

FIG. 4 shows the altered cellular accumulation and altered distribution of 3S-HS and HS3ST2 in the cell membrane and at the intracellular Level of MDM M0 from AD vs control individuals. 3S-HS enhanced immunostaining in AD patient is observed in both confocal optical slides at a z axis plane near the cell basement (FIG. 4A1), at the middle of the cell (FIG. 4B1), and reflected by the total confocal projection of the entire cell (FIG. 3C1). HS3ST2 immunostaining shows altered cellular accumulation, while in control cells HS3ST2 accumulates in the ruffling regions near the cell basement (FIG. 4A2), in AD HS3ST2 acquired a diffuse cytosolic distribution (FIG. 4B2). This intracellular cellular relocation 3S-HS is also observed in the total confocal projection of the entire cell (FIG. 4C2).

### Conclusion

Compared to control MDM M0, AD shows morphologically altered MDM M0, which accumulate 3S-HS at the intracellular level and at the cell membrane. This is accompanied by an altered cell distribution of HS3ST2, which disappears from the ruffing areas to be located at the cytosol in the AD cells. In conclusion, the differential 3S-HS levels and distribution observed in AD can be used to determine whether a patient is affected or not by AD.

### Example 3: 3S-HS Immunostaining of 5 AD Patients vs 5 Control Individuals and 5 Patients Affected by Osteoarthritis (OA) and Neurologically Normal (any Detectable Dementia)

Age of the OA Patients Chosen to Match with Age of the AD Patients

Blood from 5 different AD patients and 5 control individuals and 5 neurologically normal OA patients (all of similar ages) was collected and processed to obtain MDM M0 cells, which were immunostained for 3S-HS as in example 1. Stack images were obtained with the software CellSens from a spinning disk inverted confocal microscope (IX81 DSU Olympus, 60N.A. 1.35) coupled to an Orca Hamamatsu RCCD camera. Images were processed with the ImageJ software (W. Rasband, National Institute of Health).

### Results

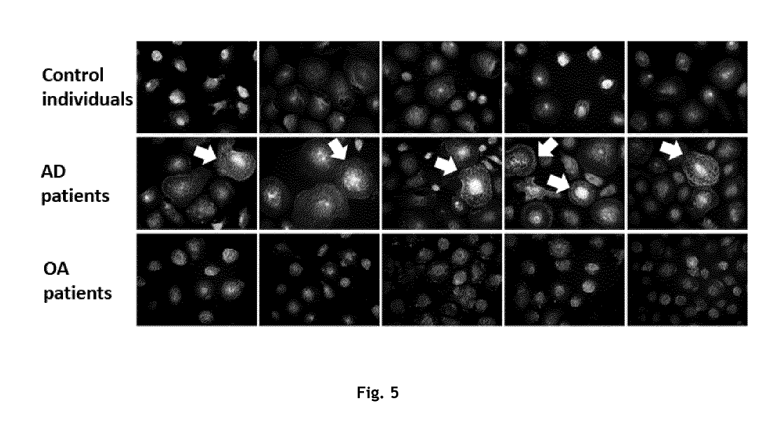

Cellular accumulation and altered distribution of 3S-HS were analyzed in MDM M0 from 5 different AD patients and 5 control individuals and 5 neurologically normal OA patients (all of similar ages). FIG. 5 total confocal projections of the entire 3S-HS immunostained cells. 3S-HS accumulated at the intracellular level and in the cell membrane of MDM M0 from AD compared to control individuals and OA and control individuals, which did not show the AD phenotype.

### Conclusion

Compared to control and OA MDM M2 from control individuals, MDM M2 from AD shows morphologically altered cells which accumulate 3S-HS at the intracellular level and at the cell membrane. This is accompanied by an altered cell distribution of HS3ST2, which disappears from the ruffing areas to be located at the cytosol in the AD cells. In conclusion, the differential 3S-HS levels and distribution observed in AD can be used to determine whether a patient is affected or not by AD. Results from OA patients indicates that the observed AD phenotype is not related to an inflammatory related condition.

### Example 4: Cell Diameter, Area and 3S-HS and HS3ST2 Immunostaining in 5 AD Patients vs 5 Control Individuals

Blood from 5 different AD patients and 5 control individuals was collected and processed to obtain MDM M0 cells, which were immunostained for 3S-HS as in example 1. Stack images were obtained with the software CellSens from a spinning disk inverted confocal microscope (IX81 DSU Olympus, 60N.A. 1.35) coupled to an Orca Hamamatsu RCCD camera.

Images were processed with the ImageJ software (W. Rasband, National Institute of Health) to obtain cell diameter (as classically do the man of the art) for an average of 50 cells for each AD patient or each control subject observed at different fields. The fields were aleatory selected. Results are shown in Table 1 (n=5). Table 1 also shows the 2D interaction surface calculated as the average area of 50 cells observed in the 2D images. This area, expressed in μm², was calculated with the ImageJ software as classically do by the person skilled in the art. This represents the average area calculated on 50 cells from each AD-patient (n=5) or control subject (n=5). Fluorescence intensities, expressed in arbitrary units (AU), for 3S-HS and HS3ST2 were obtained by the ImageJ software from the analysis of 50 cells for each AD patient (n=5) or each control subject (n=5). The fluorescence was measured in the areas of the cell concentrating the concentrated fluorescence intensity. The fields were aleatory taken (Table 1). Results were similar and consistent in the different observed fields.

### Results

Table 1 shows that MDM M0 from Alzheimer's disease patient have increased size as shown by the increased diameter and 2D interaction surface (Table 1). 3S-HS fluorescent intensity was 3 times higher in MDM M0 from Alzheimer's disease that that measured in control individuals. HS3ST2 fluorescent intensity was twice lower. The ration of the 3S-HS/HS3ST2 fluorescence shows a significate difference between AD vs control individuals. This ratio is 6 times higher in Alzheimer disease MDM M0.

TABLE 1
MDM M0 size, 2D surface interaction, and levels of fluorescence intensity of preferential
location sites for 3S-HS and HS3ST2 in AD patients and control individuals.

| | | 2D Interaction | | | 3S-HS/HS3ST2 |
|---|---|---|---|---|---|
| | Diameter | surface | 3S-HS | HS3ST2 | ratio |
| | μm | μm² | Fluorescence relative intensity (AU) | | |
| Control patients | 45.91 +/− 6.196 | 1683.93 +/− 453.33 | 1305.90 +/− 448.04 | 1147.07 +/− 327.378 | 1.14 |
| AD patients | 76.46 +/− 9.82 | 4664.52 +/− 1194.62 | 3917.68 +/− 944.12 | 613.13 +/− 112.529 | 6.39 |

### Conclusion

Results in Table 1 shows that cell diameter, 2D interaction surface, 3S-H, HS3ST2, and 3S-HS/HS3ST2 can be used to identify Alzheimer disease in MDM M0 cells. This opens to the use of any of these parameters or a combination of them in the diagnostic or prognostic of Alzheimer's disease.

### REFERENCES

Anoop A, Singh P K, Jacob R S, Maji S K. CSF Biomarkers for Alzheimer's Disease Diagnosis. Int J Alzheimers Dis. 2010; 2010.

Baird A L, Westwood S, Lovestone S. Blood-Based Proteomic Biomarkers of Alzheimer's Disease Pathology. Front Neurol. 2015; 6:236.

Ballard C, Gauthier S, Corbett A, Brayne C, Aarsland D, Jones E. Alzheimer's disease. Lancet 2011; 377 (9770):1019-31.

Bossù P, Spalletta G, Caltagirone C, Ciaramella A. Myeloid Dendritic Cells are Potential Players in Human Neurodegenerative Diseases. Front Immunol. 2015; 6:632.

Cavedo E, Lista S, Khachaturian Z, Aisen P, Amouyel P, Herholz K, Jack C R Jr, Sperling R, Cummings J, Blennow K, O'Bryant S, Frisoni G B, Khachaturian A, Kivipelto M, Klunk W, Broich K, Andrieu S, de Schotten M T, Mangin J F, Lammertsma A A, Johnson K, Teipel S, Drzezga A, Bokde A, Colliot O, Bakardjian H, Zetterberg H, Dubois B, Vellas B, Schneider L S, Hampel H. The Road Ahead to Cure Alzheimer's Disease: Development of Biological Markers and Neuroimaging Methods for Prevention Trials Across all Stages and Target Populations J Prey Alzheimers Dis. 2014; 1 (3):181-202;

Fiala M, Veerhuis R. Biomarkers of inflammation and amyloid-beta phagocytosis in patients at risk of Alzheimer disease. Exp Gerontol. 2010; 45 (1):57-63).

Goedert M, Jakes R, Spillantini M G, Hasegawa M, Smith M J, et al. Assembly of microtubule-associated protein tau into Alzheimer-like filaments induced by sulphated glycosaminoglycans. Nature 1996; 383: 550-3.

Gong C X, Iqbal K. Hyperphosphorylation of microtubule-associated protein tau: a promising therapeutic target for Alzheimer disease. Curr Med Chem. 2008; 15 (23):2321-8.

Hernandez F, Perez M, Lucas J J, Avila J. Sulfo-glycosaminoglycan content affects PHF-tau solubility and allows the identification of different types of PHFs. Brain Res. 2002; 935: 65-72.

Iqbal K, Liu F, Gong C X, Alonso Adel C, Grundke-Iqbal I. Mechanisms of tau-induced neurodegeneration. Acta Neuropathol. 2009; 118 (1):53-69.

Iqbal K, Liu F, Gong C X, Grundke-Iqbal I. Tau in Alzheimer disease and related tauopathies. Curr Alzheimer Res. 2010; 7 (8):656-64.

Mantovani A, Sica A, Sozzani S, Allavena P, Vecchi A, Locati M. The chemokine system in diverse forms of macrophage activation and polarization. Trends Immunol. 2004; 25 (12):677-86.

Mushtaq G, Greig N H, Anwar F, Zamzami M A, Choudhry H, Shaik M M, Tamargo I A, Kamal M A., miRNAs as Circulating Biomarkers for Alzheimer's Disease and Parkinson's Disease. Med Chem. 2015 Oct. 30. [Epub ahead of print PMID: 26527155].

Ballard C, Gauthier S, Corbett A, Brayne C, Aarsland D, Jones E. Alzheimer's disease. Lancet 2011; 377 (9770):1019-31.

Ritter A, Cummings Fluid Biomarkers in Clinical Trials of Alzheimer's Disease Therapeutics. J. Front Neurol. 2015; 6:186.

Sandwall E, O'Callaghan P, Zhang X, Lindahl U, Lannfelt L, Li J P. Heparan sulfate mediates amyloid-beta internalization and cytotoxicity. Glycobiology 2010; 20 (5):533-41.

Snow A D, Mar H, Nochlin D, Sekiguchi R T, Kimata K, Koike Y, et al. Early accumulation of heparan sulfate in neurons and in the beta-amyloid protein-containing lesions of Alzheimer's disease and Down's syndrome. Am J Pathol. 1990; 137 (5): 1253-70.

Su J H, Cummings B J, Cotman C W. Localization of heparan sulfate glycosaminoglycan and proteoglycan core protein in aged brain and Alzheimer's disease. Neuroscience 1992; 51 (4):801-13.

1. An in vitro method of prognosis and/or diagnosis of Alzheimer's disease, in a subject comprising the steps of:
a) isolating circulating immune cells from said subject;
b) determining the level and/or cellular distribution of heparan sulfates (HS) and/or heparan sulfate sulfotransferases (HSSTs) in said circulating immune cells;
c) comparing:
said level and/or cellular distribution of HS and/or HSSTs; or
the ratio of said level of HSSTs to said level of HS,
to a respective reference representing a known disease or health status.

2. The in vitro method according to claim 1, wherein said HS is 3-O-sulfated heparan sulfates (3S-HS), and said HSSTs are selected from heparan sulfate 3-O-sulfotransferases (HS3ST), preferentially HS3ST1, HS3ST2, HS3ST3, HS3ST4, HS3ST5 and/or HS3ST6, more preferentially HS3ST2, HS3ST3A and/or HS3ST3B.

3. The in vitro method according to claim 1, wherein said circulating immune cells are circulating T cells, B cells, monocytes and/or monocyte derived macrophages (MDM), preferentially circulating monocytes and/or MDM.

4. The in vitro method according to claim 1, wherein said method further comprises after said step a) a culturing step a1) of said circulating immune cells in an appropriate culture medium, such as RPMI 1640 medium.

5. The in vitro method according to claim 4, wherein said culturing step a1) is performed between 7-10 days, preferably 10 days, within said appropriate culture medium complemented with M-CSF until said monocytes and/or MDM present a M0 phenotype.

6. The in vitro method according to claim 5, wherein said culturing step, following said step a1), comprises an additional culturing step a2) of 2 to 3 days, preferably 3 days, in said appropriate culture medium complemented with M-CSF which further comprises anti-inflammatory factors, preferably IL4/IL10, until said M0 macrophage presents a M2 macrophage phenotype, or which further comprises pro-inflammatory factors, preferably Toll-like receptor (TLR) ligands, LPS and IFNγ, until said M0 macrophage presents a M1 macrophage phenotype.

7. The in vitro method according to claim 1, wherein said level of anyone of HS and HSSTs is determined by a method selected from Immunofluorescence, Western Blot, ELISA, mass spectrometry, flow cytometry methods, immunohistochemistry methods, and combination thereof.

8. The in vitro method according to claim 1, wherein said level and/or said cellular location of HS3ST is altered.

9. The in vitro method according to claim 1, wherein said level of HS or 3S-HS is altered and/or its cellular location is altered to be accumulated essentially into the cytosol.

10. A circulating biological marker for Alzheimer's disease consisting of at least one of HS and/or HSSTs, preferably 3S-HS and/or HS3ST, said HS3ST being preferentially selected from HS3ST1, HS3ST2, HS3ST3A, HS3ST3B, HS3ST4, HS3ST5 and/or HS3ST6, preferably HS3ST2, HS3ST3A, HS3ST3B and/or HS3ST5, for use as a circulating biological marker for Alzheimer disease.

11. A kit for the prognosis and/or diagnosis of Alzheimer's disease comprising purification means of circulating immune cells, preferably monocytes and/or monocytes derived macrophages (MDM), and detection means of level and/or cellular distribution of HS and/or HSSTs, preferably 3S-HS and/or HS3ST, said HS3ST being preferentially selected from HS3ST1, HS3ST2, HS3ST3A, HS3ST3B, HS3ST4, HS3ST5 and/or HS3ST6, preferably HS3ST2, HS3ST3A, HS3ST3B and/or HS3ST5.

12. A kit according to claim 11 comprising:
at least one primer combination to amplify said HS and/or HSSTs, preferably 3S-HS and/or HS3ST, said HS3ST being preferentially selected from HS3ST1, HS3ST2, HS3ST3A, HS3ST3B, HS3ST4, HS3ST5 and/or HS3ST6, preferably HS3ST2, HS3ST3A, HS3ST3B and/or HS3ST5; and/or
at least one probe, such as nucleic acid probes, to detect said HS and/or HSSTs, preferably 3S-HS and/or HS3ST, said HS3ST being preferentially selected from HS3ST1, HS3ST2, HS3ST3A, HS3ST3B, HS3ST4, HS3ST5 and/or HS3ST6, preferably HS3ST2, HS3ST3A, HS3ST3B and/or HS3ST5; and/or
at least one specific antibody of said HS and/or HSSTs, preferably 3S-HS and/or HS3ST, said HS3ST being preferentially selected from HS3ST1, HS3ST2, HS3ST3A, HS3ST3B, HS3ST4, HS3ST5 and/or HS3ST6, preferably HS3ST2, HS3ST3A, HS3ST3B and/or HS3ST5.

13. A kit according to claim 12 wherein said specific antibody is a conjugated antibody linked to a colorimetric or fluorescent label.

14. A kit according to claim 11, wherein said purification means comprise filters selectively retaining circulating immune cells, preferably circulating monocytes and/or monocytes derived macrophages (MDM).

15. A kit according to claim 11, wherein said kit further comprises:
Buffers, preferably Phosphate Buffered Saline;
an appropriate cell culture medium, such as RPMI 1640 medium;
M-CSF;
optionally pro and/or anti-inflammatory factors, preferably selected from recombinant human cytokines IL4 and IL10; or Toll-like receptor (TLR) ligands, LPS and IFNγ; and
one or more cell culture containers.
